(19) United States
(12) Patent Application Publication
(10) Pub. No.: US 2015/0238615 A1
(43) Pub. Date: Aug. 27, 2015

(54) Denaturants for sympathomimetic amines

(71) Applicants: Leonard MACKLES, (US); BESS William, (US); PRODUCT AND TECHNOLOGY PARTNERS LLC, Edison, NJ (US)

(72) Inventors: Leonard Mackles, New York, NY (US); William Bess, Edison, NJ (US)

(21) Appl. No.: 14/430,847

(22) PCT Filed: Nov. 20, 2012
(86) PCT No.: PCT/US2012/066018

§ 371 Date: Mar. 24, 2015

Related U.S. Application Data
(63) Continuation of application No. 13/323,713, filed on Dec. 12, 2011, now abandoned.

(51) Int. Cl. A61K 47/38; A61K 31/137

(52) U.S. Cl. CPC A61K 47/38; A61K 31/137

(57) ABSTRACT

There are provided pharmaceutical compositions comprising a sympathomimetic amine, one or more amino polymers and one or more enteric polymeric compounds insoluble in water at pH below 5.5 the combination of which acts both to interfere with the isolation of the sympathomimetic amine from the composition and to interfere with the conversion of the sympathomimetic amine to another pharmacologically active compound, all of said components being well mixed with each other.

### FIELD OF THE INVENTION

The present invention relates to pharmaceutical compositions comprising a sympathomimetic amine, one or more amino polymers and one or more enteric polymeric compounds insoluble in water at pH below 5.5 the combination of which acts both to interfere with the isolation of the sympathomimetic amine from the composition and to interfere with the conversion of the sympathomimetic amine to another pharmacologically active compound.

### DISCUSSION OF THE PRIOR ART

### Sympathomimetic Amines and the Problem of Unconventional Use

The acid salts of sympathomimetic amines are widely used active agents in over-the-counter (OTC) pharmaceuticals. As their name suggests, this class of compounds produces pharmacological effects which mimic the activation of the sympathetic nervous system. For example, the hydrochloride salt of the sympathomimetic amine pseudoephedrine is a commonly used active ingredient in OTC decongestant products. It acts by causing adrenergic nerve endings to release norepinephrine, thereby stimulating alpha and beta norepinephrine receptors, particularly of the upper respiratory tract. This, in turn, results in vasoconstriction and shrinkage of swollen tissues in the sinuses and nasal passages. Its wide usage in numerous OTC products makes it readily available and easily accessible to the general public. When used in a recommended manner for approved indications, OTC pseudoephedrine hydrochloride pharmaceuticals are safe and effective. However, a problem arises when pseudoephedrine hydrochloride-containing OTC pharmaceuticals are used in an unconventional manner. Specifically, this active ingredient from OTC products is also a convenient starting material in the production of the pharmacologically active agent methamphetamine.

Methamphetamine is a powerful stimulant of the central nervous system (CNS). One of its principle pharmacological effects is the release of high levels of the neurotransmitter dopamine which stimulates brain cells. The approved therapeutic uses for methamphetamine are very limited, and such approved uses are commonly associated with the treatment of obesity. However, methamphetamine's pharmacological effects make it a popular candidate for illegal use as a recreational drug rather than as a legitimate therapeutic agent. Methamphetamine has various street names which include “speed”, “meth” and “crank”. In its hydrochloride salt form it appears as crystals and is referred to as “ice”, “crystal” and “glass”. When taken intravenously or by smoking, methamphetamine causes a burst of intense sensation which has been described as highly pleasurable. Oral or intranasal use causes a less intense euphoric high. Methamphetamine addiction can occur quickly and is characterized by increasing frequency and dosage of the drug. The CNS effects of the drug include irritability, insomnia, confusion, tremors, hyperthermia, convulsions, anxiety, paranoia and aggressiveness. In more extreme cases methamphetamine causes heart rate and blood pressure changes which can ultimately contribute to cardiovascular collapse and death.

As a natural progression, the illegal use of methamphetamine results in a demand for access to the drug through illicit routes. These illicit routes, in turn, are often supplied by the illegal production of the compound. One of the main methods used in the illegal synthesis of methamphetamine is known as the ephedrine reduction method. This procedure is relatively simple, requiring only a few steps and a small number of reagents to carry out the chemical process. The starting material used in this method is either ephedrine or pseudoephedrine. Previously, ephedrine, an enantiomer of pseudoephedrine, was the starting material of choice in the production of illegal methamphetamine. However, since regulatory efforts in the United States have significantly reduced the ease by which ephedrine can be obtained, pseudoephedrine or its corresponding salts from OTC products have become the preferred starting point for the production of illicit methamphetamine. The wide public availability of pseudoephedrine hydrochloride in OTC pharmaceuticals allows illicit drug manufacturers easy and abundant access to a suitable starting material for clandestine methamphetamine chemistry.

Methamphetamine abuse in the United States is expanding geographically as well as in numbers among the younger population. Traditionally the illegal use of this compound was predominantly localized in the west and southwest United States. However, trafficking patterns now have been detected in areas of the country previously not known to have problems with the illicit use of this drug, namely the midwest. Furthermore, among young adults the illegal use of methamphetamine is increasing. For example, in 1997 4.4% of high school seniors had used crystal methamphetamine at least once in their lifetime—up from 2.7% in 1990. The increasing and broadening scope of illegal use of methamphetamine, combined with the decreased availability of ephedrine as a synthetic starting material for methamphetamine production, indicates the demand for OTC preparations of pseudoephedrine hydrochloride by illicit drug manufacturers will continue to escalate.

The unconventional use of OTC pseudoephedrine can be combated by limiting the accessibility of such preparations to the general public. However, this restriction adversely impacts the consumer who seeks to use properly these safe and effective drugs and who has come to rely on ready access to them without the continuous authorization of a health care professional (e.g., physicians or pharmacists). The goal of the instant invention is to significantly deter the use of OTC products in illicit drug preparations without compromising the accessibility of these products to the general public.

### Prior Attempts to Minimize Illicit Use of OTC Pharmaceuticals and Prior Art

Attempts to use the active ingredients in OTC pharmaceutical products to prepare illegal drugs is not uncommon. In response, various efforts have been made to modify pharmaceutical preparations in order to prevent this type of illicit use of these products. For example, codeine can be extracted from analgesic tablets and converted to morphine and heroin. Application WO 96/08252 disclosed codeine-containing solid dosage forms comprising components that interfere with the isolation of the active ingredient.

In the case of compositions containing sympathomimetic amines, U.S. Pat. No. 6,136,864 discloses modified pharmaceutical preparations which comprise denaturing components that complicate the use of these formulations in the illegal synthesis of drugs. The main mechanism by which such a denaturant system is effective involves creating an infeasible physical separation of the sympathomimetic amine from the formulation; this denaturant system is designed to combat widely known and used methods of preparing illegal drugs from OTC products.

By manipulating the pH of a water solution of compositions disclosed in U.S. Pat. No. 6,359,011 within a narrow specific pH range, and using a specific separation technique, it is possible to separate the amino polymer from the sympathomimetic amine, thus reducing or eliminating the denaturant effect. U.S. Pat. No. 6,359,011 discloses pharmaceutical compositions comprising a sympathomimetic amine salt and at least one combination inhibitor, the combination inhibitor which acts both to interfere with the isolation of the sympathomimetic amine from the composition and to interfere with the conversion of the sympathomimetic amine to another pharmacologically active compound. The contemplated compositions may also include reaction and separation inhibitors in any mixture to assure maximum protection against the use of the sympathomimetic amine-containing compositions for illegal drug manufacture.

The surprising advantage of the present invention over the prior art using many of the components of the present invention lies in the use of enteric coating materials. Heretofore such material have been used totally to coat pharmacologically active material, their use actually within a composition rather around it is not to be found in the art.

Enteric coatings are used for one of two purposes. The first is to protect the active ingredient (drug) from the acidic condition found in the stomach. Some drugs are not stable in the acidic stomach, or are not soluble in the acidic stomach or are altered by the acidic stomach. In such a case an enteric coating is used to protect the drug from being released in the stomach, and released only when the tablet has reached the much less acidic intestine. Once in the intestine the enteric coating, which is insoluble in the acidic solution found in the stomach, will dissolve in the intestine thus releasing the drug.

The second reason for enteric coating is to protect the stomach from drugs which can damage the stomach itself. The most common example is the use of enteric coatings for Aspirin containing products. In this case the drug itself is highly irritating to the stomach lining and thus it is desirable to avoid release of the drug in the stomach and only release the drug once it is in the intestine where the higher pH of the intestine mitigates the irritation caused by the drug.

Pseudoephedrine and the other sympathomimetic amines of the instant invention, do not fall into either of the above listed categories. The drugs are not affected by the acidic condition found in the stomach, in fact they are more stable in acidic pH than at higher alkaline pH, they are highly soluble in acidic pH, and they are not altered in any way by acidic pH. Furthermore, they have no known potential to cause irritation or damage to the stomach lining. Thus there is no rationale to use an enteric coating compound in combination with any sympathomimetic amine, either as an excipient or as a coating agent.

### SUMMARY OF THE INVENTION

The present invention addresses the problem of the use of widely available OTC sympathomimetic amine formulations in the preparation of illegal drugs by disclosing novel pharmaceutical compositions that are impractical as starting material in illegal drug synthesis. These compositions are effective in deterring both well known extraction methods as well as different and possibly more sophisticated illegal drug production methods. Furthermore, the compositions of the present invention are not compromised in their release, bioavailability or dosing frequency relative to corresponding, undenatured sympathomimetic amine preparations presently available to the consumer.

The specific improvement of the present invention is a recent discovery that the denaturant effect disclosed in U.S. Pat. No. 6,359,011 can be improved upon by the addition of an enteric polymeric compound suitable due to its insolubility in water at pH 5.5 or below. The improvement is achieved due to the fact that. by manipulating the pH of a solution of compositions disclosed in U.S. Pat. No. 6,359,011 within a narrow specific pH range, and using a specific separation technique, it is possible to separate the amino polymer from the sympathomimetic amine, thus reducing or eliminating the denaturant effect. The instant invention prevents this, with the addition of a polymer with a different solubility profile than amino polymers.

### GENERAL TERMS AND DEFINITIONS

Unless specifically designated otherwise, when used herein, it should be understood that the term “sympathomimetic amine” includes both the free amine form and the acid salts of the sympathomimetic amine.

The term “inhibitor” as used in the instant invention is any component, added to a composition containing a sympathomimetic amine, which interferes with the isolation of that sympathomimetic amine and/or interferes with the conversion of that sympathomimetic amine to another pharmacologically active compound. A single inhibitor of any class (i.e., combination, reaction, enteric polymeric compound or separation) or any mixture of more than one inhibitor of the same or of multiple classes of inhibitors will not significantly effect the release of the sympathomimetic amine from the formulation as compared to the undenatured composition. The term inhibitor is interchangeable with the term “denaturant”. A “pharmacologically active compound” is any chemical substance that affects living tissue, producing a biological effect in a living organism.

The terms “immediate release” and “modified release” are used in the instant invention as they are commonly understood in the pharmaceutical industry. For immediate release products in solid dosage form (such solid dosage forms including but not limited to tablets, capsules, powders and films), release is defined as the amount of sympathomimetic amine measured using an appropriate USP dissolution test procedure with distilled water as the medium. If no official USP dissolution test procedure has been established, (e.g., as in the case of chewable tablets), then the most appropriate USP dissolution test will apply, utilizing distilled water or pH buffered distilled water as appropriate as the medium and taking measurements at appropriate time points up to and including the final dosing interval. For immediate release products in non-solid dosage form (such non-solid dosage forms including but not limited to liquids, syrups, elixers, liquid center oral products, creams, pastes and gels), release is defined as the amount of sympathomimetic amine measured after mixing a 1% solution of the product in distilled water at 37.degree. C. for 30 minutes.

For modified release products in solid dosage form (such solid dosage forms including but not limited to tablets, capsules, powders and films), release is defined as the amount of sympathomimetic amine measured using an appropriate USP dissolution test procedure with distilled water or pH buffered distilled water as appropriate as the medium. If no official USP dissolution test procedure has been established, (e.g., as in the case of chewable tablets), then the most appropriate USP dissolution test will apply, utilizing distilled water or pH buffered distilled water as appropriate as the medium and taking measurements at appropriate time points up to and including the final dosing interval. For modified release products in non-solid dosage form (such non-solid dosage forms including but not limited to liquids, syrups, elixers, liquid center oral products, creams, pastes and gels), release is defined as the amount of sympathomimetic amine measured after mixing a 1% solution of the product in distilled water at 37.degree. C. for 1 hour and taking at least one additional measurement up to and including the final dosing interval.

For the purposes of this invention, the term “combination inhibitor” refers to a component of a composition which both interferes with the isolation of a sympathomimetic amine from the composition and interferes with the conversion of the sympathomimetic amine to another pharmacologically active compound. The combination inhibitor interferes with such a conversion either directly from the original formulation or after attempts have been made to isolate the sympathomimetic amine. It is both the physical and chemical properties of the combination inhibitors which allow them to act as multifaceted deterrents to the use of these compositions in the preparation of other pharmacologically active compounds. Combination inhibitors of the instant invention demonstrate all of the following characteristics: (1) they significantly interfere with chemical reactions which convert the sympathomimetic amine to another pharmacologically active compound; (2) they are not readily separable from the sympathomimetic amine to an extent that they would no longer be able to significantly interfere with chemical reactions which convert the sympathomimetic amine to another pharmacologically active compound; and, (3) they may also interfere with the separation of the sympathomimetic amine from the other components of the composition. A single combination inhibitor may be used or multiple combination inhibitors may be included in the composition of the instant invention.

In the present invention “reaction inhibitors” are components of a composition that mainly interfere with the conversion of sympathomimetic amines to other pharmacologically active compounds. Unlike combination inhibitors, under certain conditions a reaction inhibitor may be readily separable from the sympathomimetic amine to the extent that it no longer can significantly effect the chemical reactions which convert the sympathomimetic amine to another pharmacologically active compound. A single reaction inhibitor may be used or multiple reaction inhibitors may be included in the composition of the instant invention.

A “separation inhibitor” in the instant invention is a component of a composition that primarily interferes with the separation of the sympathomimetic amine from the composition. The interference with the separation is predominately due to the inability of the sympathomimetic amine to be physically isolated from the composition. Separation inhibitors are of two main types: water soluble and solvent soluble. An example of a typical separation technique which may be affected by the properties of a separation inhibitor involves the use of filtration as a means of separating the sympathomimetic amine from the composition. In the case of filtration, the separation inhibitor interferes with the filtration and results in a reduced yield of separated sympathomimetic amine. Water soluble inhibitors interfere with water based separations and solvent soluble inhibitors interfere with solvent based separations. A third class of separation inhibitors can also be used in the present invention. This class includes compounds that are soluble in both aqueous and organic solvents. A single separation inhibitor may be used or multiple separation inhibitors may be included in the composition of the instant invention.

Sympathomimetic Amines in OTC Pharmaceuticals and the Corresponding Pharmacologically Active Compounds Produced from the Sympathomimetic Amines

Sympathomimetic amines are compounds that cause vasoconstriction in the vascular bed of the nasal mucosa resulting in a shrinking of the engorged mucous membranes. The ultimate physiological response is increased drainage and improved nasal air flow. As a result of these effects, sympathomimetic amines are highly effective as nasal decongestants. Unless otherwise stated, as used herein the term “sympathomimetic amine” can be used interchangeably with and may refer to a corresponding pharmaceutically acceptable acid salt form of the amine. The amine and its acid salt form may have asymmetric centers and occur as racemates, racemic mixtures, individual diastereomers, or enantiomers, with all isomeric forms being included in the present invention.

Those sympathomimetic amines widely available as OTC nasal decongestants are particularly contemplated for use in the instant invention; specific preferred examples include: pseudoephedrine hydrochloride, pseudoephedrine sulfate, ephedrine hydrochloride and phenylpropanolamine hydrochloride. The sympathomimetic amine phenylephrine hydrochloride is also contemplated in the instant invention. While the descriptions and examples of the compositions comprising combination inhibitors, reaction inhibitors and separation inhibitors may use a specific sympathomimetic amine such as pseudoephedrine hydrochloride, it is understood that the inhibitors are applicable to any composition comprising sympathomimetic amines.

Sympathomimetic amines in OTC preparations can be converted to a wide variety of other pharmacologically active compounds. In the instant invention the pharmacologically active compounds contemplated are ones that are used for recreational purposes in an illicit manner. Examples of such pharmacologically active compounds produced from sympathomimetic amines include, but are not limited to: methamphetamine, amphetamine, methcathinone and cathinone. The production of these illegal drugs occurs by various reductive or oxidative reactions widely known to those skilled in the art.

### DETAILED DESCRIPTION OF THE INVENTION

### The Amount of Sympathomimetic Amine Relative to the Amount of Inhibitors in the Invention.

The ratio of sympathomimetic amine salts to the inhibitor components in any composition of the instant invention is from about 1:100 to about 100:1, preferably from about 1:10 to about 10:1. Even more preferably, the amount of sympathomimetic amine salts and inhibitor mixture in any composition of the present invention is in a ratio of about 1:5 to about 5:1.

The amount of inhibitor needed to effectively interfere with the isolation of the sympathomimetic amine and the conversion of the sympathomimetic amine to another pharmacologically active compound from a composition is dependent upon the size of the unit dose. For immediate release products, typical dosages (milligrams, mg) for commonly used sympathomimetic amine decongestants are 60 mg for pseudoephedrine hydrochloride, 10 mg for phenylephrine hydrochloride, 25 mg for phenylpropanolamine hydrochloride and 24 mg for ephedrine hydrochloride. Generally, the total quantity of the inhibitors per unit dose preparation may be varied or adjusted from about 0.1 mg to about 750 mg according to the particular application and the potency of the sympathomimetic amine salt. For example, when a 30 mg pseudoephedrine hydrochloride tablet is being prepared, the amount of the inhibitor components is in a range from about 0.3 mg to about 3000 mg, more preferably in a range from about 3 mg to about 300 mg and most preferably in a range from about 6 mg to about 150 mg. The specific amounts used in the compositions can be readily determined by one of ordinary skill in the art of pharmaceutical formulations.

The goal of the instant invention is an OTC pharmaceutical composition of a sympathomimetic amine which is not usable in illicit drug preparation. The pharmaceutical composition of the present invention comprises amounts of inhibitors sufficient to interfere with both the isolation of the sympathomimetic amine and the use of the sympathomimetic amine in chemical reactions without compromising the release, bioavailability or dosing frequency associated with the undenatured composition.

### Combination Inhibitors

Combination inhibitors are components which interfere with the use of compositions of sympathomimetic amines in the production of other pharmacologically active compounds by both interfering with the isolation of the sympathomimetic amine from the composition and interfering with the conversion of the sympathomimetic amine to other pharmacologically active compounds. The presence of the combination inhibitor does not significantly alter the release of the sympathomimetic amine from the composition as compared to the undenatured composition. In the instant invention a preferred combination inhibitor may be an amino polymer or the corresponding neutralized salt form of the amino polymer. The amino polymer, in both the amine and neutralized salt forms, has a similar solubility profile to the corresponding form of many sympathomimetic amines, making it very difficult to separate the amine from a composition containing an amino polymer.

Additionally, the amino polymer inhibits the chemical conversion of sympathomimetic amines to other pharmacologically active compounds. The prior art teaches the use of unneutralized amino polymers as coating agents for sympathomimetic amines (or other pharmacologically active agents). In such references the purpose of the uneutralized amino polymer in the composition is to prevent the active ingredient (e.g., a sympathetic amine) from dissolving in the mouth and creating an undesirable taste. Thus, the unneutralized amino polymer is used in a manner specifically intended to modify the release of the coated active agent in water as compared to the uncoated active ingredient. However, upon reaching the stomach, the gastric fluid pH will neutralize the amino polymer, thus insuring that the sympathomimetic amine will be released from the amino polymer coating. In the instant invention the use of the unneutralized amino polymer may have an effect on the release of sympathomimetic amine in water, but will not affect its release in the stomach, thus insuring the desired pharmacologic effect.

One example of an amino polymer contemplated in this invention is a copolymer of methyl methacrylate, butyl methacrylate and dimethylaminoethyl methacrylate, also known as aminoalkyl methacrylate copolymer E, JP. A preferred copolymer of methyl methacrylate, butyl methacrylate and dimethylaminoethyl methacrylate is Eudragit-E. In the instant invention this amino polymer is utilized in a neutralized form. A second example of an amino polymer is chitosan, an amino-polysaccharide polymer which may be used either neutralized or unneutralized.

### Enteric Polymeric Compounds

Three classes of enteric polymeric compounds are suitable for use for this purpose.

The first class consists of polymers of cellulose acetate phthalate. Cellulose acetate phthalate is normally utilized as an enteric coating for pharmacologically active compounds because it is not soluble in water below pH 5.5. Hence it does not release the pharmacologically active compound in the gastric fluid of the stomach, but does release it in the higher pH (above 5.5) of the intestine.

The second class is copolymers of ethylacrylate and methacrylic acid which are insoluble in water at a pH below 5.5. U.S. Pat. No. 6,359,011 discloses the use of “acrylic acid polymers such as Carbomer” as separation inhibitors. These acrylic acid polymers differ from the ethylacrylate copolymer in the instant invention in that they are water soluble at pH below 5.5, are crosslinked, and do not prevent the separation of the amino polymer and sympathomimetic amine as described above. The instant invention utilizes a linear non-crosslinked co-polymer of ethylacrylate and methyacrylic acid which is insoluble in water at pH below 5.5, and prevents the separation of amino polymer and sympathomimetic amine as described above.

The third class is Hypromellose-Acetate-Succinate. Hypromellose-Acetate-Succinate is normally utilized as an enteric coating for pharmacologically active compounds because it is not soluble in water below pH 5.5. Hence it does not release the pharmacologically active compound in the gastric fluid of the stomach, but does release it in the higher pH (above 5.5) of the intestine.

The enteric polymeric compounds being water insoluble at pH below 5.5 are normally utilized to coat pharmacologically active compounds in order to modify the release of said compound by bypassing the stomach and releasing it in the intestines. In the instant invention the use of such compounds will not have any effect on the release of the sympathomimetic amine as compared to its release from the same product without the enteric polymeric compound. A preferred copolymer of ethylacrylate and methacrylic acid is Eudragit L-100-55. A preferred polymer of cellulose acetate phthalate is Eastman C-A-P powder. A preferred polymer of Hypromellose-Acetate-Succinate is Shin-Etsu AQOAT A single enteric polymeric compound may be used or multiple enteric polymeric compounds may be included in the composition of the instant invention.

In one embodiment of the instant invention it is contemplated that the amino polymer is homogeneously mixed together with the enteric polymeric compound and the sympathomimetic amine and any other components necessary to prepare the final composition. In another embodiment the sympathomimetic amine is first coated with the amino polymer prior to combining these two components with the enteric polymeric compound and any additional components required in the formulation.

### Reaction Inhibitors

A preferred embodiment of the instant invention is a pharmaceutical composition comprising an acid salt of a sympathomimetic amine, an enteric polymeric compound, an amino polymer and at least one reaction inhibitor. The amino polymer is present in amounts sufficient to interfere with both the isolation of the amine and the conversion of the amine to another pharmacologically active compound; the enteric polymeric compound is in an amount sufficient to prevent separation of the amino polymer from the sympathomimetic amine; the reaction inhibitor is present in amounts sufficient to interfere with the conversion of the amine to another pharmacologically active compound. The presence of the amino polymer, the enteric polymeric compound and reaction inhibitors does not significantly alter the release of the sympathomimetic amine from the composition as compared to the undenatured composition when ingested.

Reaction inhibitors useful in the instant invention include the following classes of agents of which examples of preferred compounds are listed: (1) water insoluble polyhydroxy compounds such as cellulose, ethylcellulose and microcrystalline cellulose; (2) non-polymeric (three or fewer repeating units) water soluble polyhydroxy compounds such as glycerin, sucrose, lactose, fructose, sorbitol, lactitol, maltose, other mono- and disaccharides and other sugar alcohols; and, (3) solvent soluble esters such as glycerin esters, esters of glycerin polymers, sorbitol esters, propylene glycol esters, polyethylene glycol esters, sucrose esters and esters of ethoxylated fatty acids, and ingestible transition metal compounds such as ferrous gluconate, zinc gluconate, and copper gluconate.

The compositions according to this invention can include a single reaction inhibitor that is soluble in water, methanol, chloroform and/or other organic solvents; alternatively, the reaction inhibitor can be a mixture consisting of a group of reaction inhibitors that individually are soluble in one or more solvents but have a combined solubility profile such that they cover a range of aqueous and organic solubilities. The use of such a reaction inhibitor mixture provides a high likelihood that at least one reaction inhibitor remains with the sympathomimetic amine to interfere with subsequent chemical reactions if a separation step is successful. An example of a reaction inhibitor mixture that has combined solubility in water, methanol and chloroform could contain lactose and ethylcellulose. In one embodiment of the instant invention, at least one reaction inhibitor is utilized.

### Separation Inhibitors

Separation inhibitors are compounds which interfere with or inhibit the separation of the sympathomimetic amine from other constituents of the composition. Separation inhibitors useful in the instant invention include the following classes of agents of which examples of the preferred compounds are listed: (1) water soluble celluloses such as hydroxypropyl cellulose, methylcellulose and hydroxyethycellulose; (2) polysaccharide gums such as guar and xanthan; (3) polyethylene oxide polymers such as polyethylene oxide, poly(oxypropylene)-poly(oxyethylene) block polymers and polyethylene glycols; (4) acrylic acid polymers such as carbomers; (5) starches such as pre-gellatinized starch, corn starch and potato starch; (6) magnesium aluminum silicates such as Veegum; (7) polyvinylpyrrolidones; and (8) clays such as Kaolin and Bentonite. As an example, in an immediate release product separation inhibitors may be used in amounts (milligrams, mg) per unit dose from about 0.1 mg to about 100 mg, more preferably from about 0.5 mg to about 75 mg and most preferably from about 1 mg to about 50 mg. A preferred embodiment of a separation inhibitor in the instant invention comprises a separation inhibitor mixture comprising hydroxypropyl cellulose, hydroxyethylcellulose, and hydroxypropyl methylcellulose.

### Physical Appearance

It is desirable wherever possible to match the physical appearance of any inhibitor or combination of inhibitors to that of the physical appearance of the sympathomimetic amine. This will prevent the physical separation of the various components of the formula by such means as screening, overmixing to achieve blend segregation and other physical separation techniques. Non-limiting examples of physical appearance include, color, particle shape, and particle size. For example, in a delayed release capsule, the sympathomimetic amine may be in the form of a coated bead designed to control the release of the sympathomimetic amine. The denaturants should also be in the same form, shape and color as the beads containing the sympathomimetic amine. This will prevent the physical separation of the various beads by use of screens, or other physical separation techniques.

### EXAMPLES

### Example 1

### Capsule Formulation

| Ingredients | Weight per Dose |
|---|---|
| Pseudoephedrine HCl | 30.0 mg |
| Eudragit E HCl | 20.0 mg |
| Eudragit L-100-55 | 10.0 mg |
| Ethylcellulose | 10.0 mg |
| Sucrose | 20.0 mg |
| Hydroxypropyl Methylcellulose | 3.0 mg |

The ingredients are combined and mixed until uniform then encapsulated in a suitable hard gelatin capsule.

### Example 2

### Capsule Formulation without Chitosan

| Ingredient | Weight per dose |
|---|---|
| Pseudoephedrine HCl | 90.0 mg |
| Eudragit E HCl | 50.0 mg |
| Cellulose Acetate Phthalate | 25.0 mg |
| Ethylcellulose | 10.0 mg |
| Xanthan Gum | 5.0 mg |
| Chitosan | 30.0 mg |
| Fructose | 15.0 mg |
| Hydroxypropyl Cellulose | 2.0 mg |

The ingredients are mixed until uniform and encapsulated in a suitable hard gelatin capsule.

### Example 3

### Capsule Formulation with Chitosan

| Ingredient | Weight per dose |
|---|---|
| Pseudoephedrine HCl | 30.0 mg |
| Chitosan | 30.0 mg |
| Eudragit E HCl | 10.0 mg |
| Eudragit L-100-55 | 10.0 mg |
| Ferrous Gluconate | 10.0 mg |
| Copper Gluconate | 5.0 mg |
| Zinc Gluconate | 10.0 mg |
| Sorbitol | 20.0 mg |
| Carboxymethylcellulose | 5.0 mg |

The ingredients are mixed until uniform and encapsulated in a suitable hard gelatin capsule.

### Example 4

### Compression Tablet with Chitosan

| Ingredients | Weight per Dose |
|---|---|
| Pseudoephedrine HCl | 60.0 mg |
| Chitosan | 20.0 mg |
| Eudragit E HCl | 60.0 mg |
| Cellulose Acetate Phthalate | 20.0 mg |
| Ethylcellulose | 60.0 mg |
| Hydroxypropyl Cellulose | 10.0 mg |
| Lactose | 60.0 mg |
| Fructose | 60.0 mg |
| Ferrous Gluconate | 1.0 mg |
| Crosspovidone | 5.0 mg |
| Stearic Acid | 2.5 mg |
| Mg Stearate | 1.0 mg |
| Silicon Dioxide M-5 | 4.0 mg |

60 mg pseudoephedrine HCl USP/EP, 10 mg hydroxypropyl cellulose NF and 60 mg ethylcellulose NF are combined and mixed well. This mixture is then thermally extruded to produce a granulation. 60 mg of aminoalkyl methacrylate copolymer E HCl and 20 mg. cellullose acetate phthalate are added to the granulate and sized through an appropriate mill

4 mg of silicon dioxide NF/EP, 60 mg fructose NF, 60 mg lactose NF 1 mg ferrous gluconate, and 5 mg crospovidone NF and 20 mg chitosan are delumped and combined with the sized material and blended until uniform.

1.5 mg stearic acid NF and 0.5 mg magnesium stearate NF are sifted into the blend produced and the resultant blend compressed on a standard press to the desired weight and thickness.

### Example 5

### Compression Formulation without Chitosan

| Ingredients | Weight per Dose |
|---|---|
| Pseudoephedrine HCl | 30.0 mg |
| Eudragit E HCl | 30.0 mg |
| Hypromellose Acetate Succinate | 10.0 mg |
| Ethylcellulose | 20.0 mg |
| Hydroxypropyl Cellulose | 5.0 mg |
| Fructose | 30.0 mg |
| Ferrous Gluconate | 1.0 mg |
| Crosspovidone | 2.5 mg |
| Stearic Acid | 1.5 mg |
| Mg Stearate | 0.5 mg |
| Silicon Dioxide M-5 | 2.0 mg |

30 mg pseudoephedrine HCl USP/EP, 5 mg hydroxypropyl cellulose NF and 20 mg ethylcellulose NF are combined and mixed well. This mixture is then thermally extruded to produce a granulation. 30 mg of aminoalkyl methacrylate copolymer E HCl and 10 mg. hypromellose acetate succinate are added to the granulate and sized through an appropriate mill.

2 mg of silicon dioxide NF/EP, 30 mg fructose NF, 1 mg ferrous gluconate, and 2.5 mg crospovidone NF are delumped and combined with the sized material and blended until uniform.

1.5 mg stearic acid NF and 0.5 mg magnesium stearate NF are sifted into the blend produced and the resultant blend compressed on a standard press to the desired weight and thickness.

### Example 6

### Compression Formulation

| Ingredient | Weight per dose |
|---|---|
| Pseudoephedrine HCl | 30.0 mg |
| Eudragit E HCl | 30.0 mg |
| Cellulose Acetate Phthalate | 10.0 mg |
| Ethylcellulose | 20.0 mg |
| Polyethylene Oxide | 5.0 mg |
| Sorbitol | 15.0 mg |
| Zinc Gluconate | 12.5 mg |
| Crosspovidone | 2.5 mg |
| Stearic Acid | 1.5 mg |
| Mg Stearate | 0.5 mg |
| Silicon Dioxide M-5 | 2.0 mg |

30 mg pseudoephedrine HCl USP/EP, and 20 mg ethylcellulose NF are combined and mixed well. This mixture is then thermally extruded to produce a granulation. 30 mg of aminoalkyl methacrylate copolymer E HCl and 10 mg. cellullose acetate phthalate and 5 mg. of polyethylene oxide. are added to the granulate and sized through an appropriate mill.

2 mg of silicon dioxide NF/EP, 15 mg sorbitol, 15 mg zinc gluconate, and 2.5 mg crospovidone NF are delumped and combined with the sized material and blended until uniform.

1.5 mg stearic acid NF and 0.5 mg magnesium stearate NF are sifted into the blend produced and the resultant blend compressed on a standard press to the desired weight and thickness.

We claim:

1. A pharmaceutical composition comprising:
an acid salt of a sympathomimetic amine; and
at least one combination inhibitor, said combination inhibitor consisting of an amino polymer, said combination inhibitor is present in amounts sufficient to interfere with the isolation of said sympathomimetic amine and to interfere with the conversion of said sympathomimetic amine to other pharmacologically active compounds without significantly altering the release of said sympathomimetic amine from said pharmaceutical composition as compared to the undenatured composition; and
an enteric polymeric compound which is insoluble in water at pH below 5.5 all of said components being well mixed with each other

2. The pharmaceutical composition according to claim 1 in which the enteric polymeric compound is cellulose acetate phthalate.

3. The pharmaceutical composition according to claim 1 in which the enteric polymeric compound is a copolymer of ethylacrylate and methacrylic acid which is insoluble in water at pH below 5.5.

4. The pharmaceutical composition according to claim 1 in which the enteric polymeric compound is hypromellose acetate succinate.

5. The pharmaceutical composition according to claim 1, further comprising at least one reaction inhibitor, wherein said reaction inhibitor is present in amounts sufficient to interfere with the conversion of said sympathomimetic amine to other pharmacologically active compounds without significantly altering the release of said sympathomimetic amine when ingested from said pharmaceutical composition as compared to the undenatured composition.

6. The pharmaceutical composition according to claim 1, further comprising at least one separation inhibitor, wherein said separation inhibitor is present in amounts sufficient to interfere with the isolation of said sympathomimetic amine without significantly altering the release of said sympathomimetic amine when ingested from said pharmaceutical composition as compared to the undenatured composition.

7. The pharmaceutical composition according to claim 1, in which at least one inhibitor has the same physical appearance as the sympathomimetic amine.

8. The pharmaceutical composition according to claim 5 wherein the reaction inhibiter is an ingestible transition metal compound.

9. The pharmaceutical composition according to claim 1 wherein said sympathomimetic amine is selected from the group consisting of pseudoephedrine hydrochloride, pseudoephedrine sulfate, ephedrine hydrochloride and phenylpropanolamine hydrochloride.

10. The pharmaceutical composition according to claim 6 wherein said sympathomimetic amine is pseudoephedrine hydrochloride.

11. The pharmaceutical composition according to claim 1 wherein said other pharmacologically active compound is selected from the group consisting of methamphetamine, amphetamine, methacathinone and cathinone.

12. The pharmaceutical composition according to claim 8 wherein said other pharmacologically active compound is methamphetamine.

13. The pharmaceutical composition according to claim 1, wherein said amino polymer is in a neutralized form.

14. The pharmaceutical composition according to claim 1 wherein said amino polymer is a copolymer of methyl methacrylate, butyl methacrylate and dimethylaminoethyl methacrylate.

15. A process for the formation of a product of claim 1 wherein all components are mixed together.

16. The process of claim 15 wherein the sympathomimetic amine is first coated with the amino polymer prior to combining these two components with the enteric polymeric compound.

17. The process of claim 16 wherein the sympathomimetic amine is first coated with the amino polymer prior to combining these two components with the enteric polymeric compound and any additional components required in the formulation.
